(19) United States
(12) Patent Application Publication
(10) Pub. No.: US 2025/0262256 A1
(43) Pub. Date: Aug. 21, 2025

(54) BACTERIAL STRAIN COMPOSITION FOR INCREASING ENTEROCHROMAFFIN CELL NUMBER AND REDUCING CORTISOL LEVEL

(71) Applicant: GLAC BIOTECH CO., LTD, Tainan (TW)

(72) Inventors: Hsieh-Hsun HO, Tainan (TW); Ko-Chiang HSIA, Tainan (TW); Jia-Hung LIN, Tainan (TW); Yi-Wei KUO, Tainan (TW); Shin-Yu TSAI, Tainan (TW); YU-CHIEH HSU, Tainan (TW); Yen-Yu HUANG, Tainan (TW); Ching-Min LI, Tainan (TW)

(21) Appl. No.: 19/051,381

(22) Filed: Feb. 12, 2025

(30) Foreign Application Priority Data
Feb. 16, 2024 (TW) 113105574

(51) Int. Cl. A61K 35/747; A61K 35/745

(52) U.S. Cl. CPC A61K 35/747; A61K 35/745

(57) ABSTRACT

The present invention provides a bacterial strain composition for increasing enterochromaffin cell number and reducing cortisol level, which comprises: a Bifidobacterium breve CCFM1025 strain, a Lactobacillus plantarum LPL28 strain, and a Lactobacillus acidophilus TYCA06 strain; wherein the CCFM1025 strain is deposited at the Guangdong Microbial Culture Center with a deposition number of GDMCC 60386; the LPL28 strain is deposited at the China General Microbiological Culture Collection Center with a deposition number of CGMCC 17954; the TYCA06 strain is deposited at the China General Microbiological Culture Collection Center with a deposition number of CGMCC 15210.

### CROSS REFERENCE

This non-provisional application claims priority of Taiwan Invention Patent Application No. 113105574, filed on Feb. 16, 2024, the contents thereof are incorporated by reference herein.

### FIELD OF THE INVENTION

The present invention is related to a bacterial strain composition, and more particularly to a bacterial strain composition for increasing enterochromaffin cell number and reducing cortisol level.

### BACKGROUND OF THE INVENTION

Mood swing is influenced by various complicated factors, e.g., physiology factors, psychological factors, and environmental factors. During stressful events, the adrenal gland can secrete cortisol to facilitate an appropriate response. However, as a cortisol level in the body increase, feelings of anxiety, tension, and unease may also arise. Serotonin is a neurotransmitter, and is also one of the main emotion regulating agents for the human body. It is well known that serotonin is related to emotional stability, sleep quality, concentration, and happiness. More than 90% of serotonin in the body is synthesized in the intestinal tract, wherein enterochromaffin cells are the cells for synthesizing, storing, and secreting serotonin in the body. Specifically, with persistent elevation of the cortisol level or persistent decline of the serotonin level in the body, the human may develop physical and mental symptoms, e.g., anxiety, irritability, depression, emotional instability, metabolic syndrome, Cushing's syndrome, attention deficit, or sleep disorder.

The “gut-brain axis” is a communication bridge between the brain and the intestinal tract and involves numerous mechanisms, e.g., vagus nerves, microbial metabolism, hormone, immunoregulation, and hypothalamus-pituitary-adrenal axes (HPA). Therefore, the “gut-brain axis” further has an effect on cognition and emotion. According to J Physiol. 2004 Jul. 1; 558 (Pt 1):263-75, N. Sudo et al. discover that plasma adrenocorticotropic hormone (ACTH) and corticosterone elevation in response to restraint stress is substantially higher in germ-free mice than in specific pathogen-free mice. The exaggerated hypothalamus-pituitary-adrenal axis stress response by germ-free mice is reversed by reconstitution with Bifidobacterium infantis. In contrast, the colonization of enteropathogenic E. coli enhances the response to hypothalamus-pituitary-adrenal axis stress. These results indicate that the composition of the gut microbiota plays a crucial role in the body's response to stress. A beneficial gut microbiota composition can influence the stress response by regulating changes in the hypothalamus-pituitary-adrenal axis.

Lactic acid bacteria are generally recognized as safe, familiar, and widely used probiotics, and the common genera include: Lactobacillus, Lactococcus, Pediococcus, Enterococcus, Streptococcus, Bifidobacterium, Bacillus, and Leuconostoc. In Clin Nutr. 2019 April; 38(2):522-528, A. Kazemi et al. investigate the effect of lactic acid bacteria supplement, e.g., Lactobacillus helveticus or Bifidobacterium longum, on patients suffering from major depression. As compared with the control group, the group treated with lactic acid bacteria have relatively low Beck depression inventory score, and have cortisol concentration 20% lower than the baseline. These results indicate that oral administration of lactic acid bacteria can improve the depression symptoms of patients suffering from major depression. In light of Front Microbiol. 2023 May 10:14:1174800, J. Li et al. find Bifidobacterium longum R0175 strain, Bifidobacterium infantis 35624 strain, Lactobacillus helveticus R0052 strain, Bifidobacterium adolescentis NK98 strain, Bifidobacterium breve M-16V strain, and Bifidobacterium breve M2CF22M7 strain all have a positive effect on treating depression, and the mechanism of anti-depressant action may be associated with regulating inflammatory response, tryptophan metabolism, 5-hydroxytryptamine synthesis, hypothalamus-pituitary-adrenal axes by the gut-brain axis.

As described above, lactic acid bacteria are generally safe, and therefore there is a need to develop a bacterial strain composition which can be taken for a long term for regulating emotion and relieving anxiety.

### SUMMARY OF THE INVENTION

Herein, it is discovered that the combination of a Bifidobacterium breve CCFM1025 strain, a Lactobacillus plantarum LPL28 strain, and a Lactobacillus acidophilus TYCA06 strain can increase the enterochromaffin cell number and reduce the cortisol level. Therefore, the combination can improve anxiety and stress behavior and is thus expected to reduce the subject's stress.

An aspect of the present invention provides a bacterial strain composition for increasing enterochromaffin cell number and reducing cortisol level, which comprises: a Bifidobacterium breve CCFM1025 strain, a Lactobacillus plantarum LPL28 strain, and a Lactobacillus acidophilus TYCA06 strain; wherein the CCFM1025 strain is deposited at the Guangdong Microbial Culture Center with a deposition number of GDMCC 60386; the LPL28 strain is deposited at the China General Microbiological Culture Collection Center with a deposition number of CGMCC 17954; the TYCA06 strain is deposited at the China General Microbiological Culture Collection Center with a deposition number of CGMCC 15210.

Another aspect of the present invention provides a method for increasing enterochromaffin cell number and reducing cortisol level, which comprises: administering to a subject in need thereof a composition comprising: a Bifidobacterium breve CCFM1025 strain, a Lactobacillus plantarum LPL28 strain, and a Lactobacillus acidophilus TYCA06 strain; wherein the CCFM1025 strain is deposited at the Guangdong Microbial Culture Center with a deposition number of GDMCC 60386; the LPL28 strain is deposited at the China General Microbiological Culture Collection Center with a deposition number of CGMCC 17954; the TYCA06 strain is deposited at the China General Microbiological Culture Collection Center with a deposition number of CGMCC 15210.

### BRIEF DESCRIPTION OF THE DRAWINGS

The above and other objects, features and other advantages of the present invention and examples will be more clearly understood in conjunction with the accompanying drawings, in which:

FIG. 1 is a bar graph illustrating mouse's time spent in the dark room in the light/dark box test, wherein * indicates p<0.05, and **** indicates p<0.0001;

FIG. 2 is a bar graph illustrating enterochromaffin cell number in mouse's ileum, wherein * indicates p<0.05, and *** indicates p<0.001;

FIG. 3 is a bar graph illustrating mouse's serum tryptophan level, wherein ** indicates p<0.01, and **** indicates p<0.0001;

FIG. 4 is a bar graph illustrating mouse's serum 5-hydroxytryptamine level, wherein *** indicates p<0.001, and **** indicates p<0.0001;

FIG. 5 is a bar graph illustrating mouse's serum cortisol level, wherein ** indicates p<0.01, and *** indicates p<0.001;

FIG. 6 is a bar graph illustrating relative number of rat pancreatic islet tumor RIN-14B cells; and

FIG. 7 is a bar graph illustrating cortisol relative level of human adrenocortical HAC15 cells.

### DETAILED DESCRIPTION OF THE INVENTION

The detailed description and preferred embodiments of the invention will be set forth in the following content, and provided for people skilled in the art to understand the characteristics of the invention.

The present invention provides a bacterial strain composition, which can increase enterochromaffin cell number and reduce cortisol level, and the bacterial strain composition comprises: a Bifidobacterium breve CCFM1025 strain, a Lactobacillus plantarum LPL28 strain, and a Lactobacillus acidophilus TYCA06 strain; wherein the CCFM1025 strain is deposited at the Guangdong Microbial Culture Center with a deposition number of GDMCC 60386; the LPL28 strain is deposited at the China General Microbiological Culture Collection Center with a deposition number of CGMCC 17954; the TYCA06 strain is deposited at the China General Microbiological Culture Collection Center with a deposition number of CGMCC 15210.

In an embodiment, the bacterial strain composition is a culture containing the CCFM1025 strain, the LPL28 strain, and the TYCA06 strain. The so-called “culture” is obtained by incubating the CCFM1025 strain, the LPL28 strain, and the TYCA06 strain in a suitable medium, respectively. The suitable medium is well known to the skilled in this art, and may be self-prepared or commercially purchased. The suitable medium comprises, but not limited to, an MRS broth medium, an MRS broth medium containing cysteine, a medium containing glucose, yeast extract, and soy peptone, an LBS medium, or an LBS medium containing cysteine. The conditions and parameters for incubation are determined based on the expertise and the routine of the skilled in this art and chosen with reference to New Microbiol. 2013 April; 36(2):167-79. In an embodiment, the incubation is performed in a temperature from 25° C. to 40° C. for a period from 24 hrs to 48 hrs, and preferably in a temperature of 37° C. for a period of 24 hrs.

In an embodiment, based on the total volume of the culture, total bacterial concentration of the CCFM1025 strain, the LPL28 strain, and the TYCA06 strain is from 10⁶ CFU/mL to 10¹² CFU/mL, preferably from 10⁶ CFU/mL to 10¹⁰ CFU/mL, and more preferably 10⁹ CFU/mL.

In an embodiment, the CCFM1025 strain, the LPL28 strain, and the TYCA06 strain are separately a viable bacterial strain or a deactivated bacterial strain. In an embodiment, the composition is concentrated or non-concentrated, is a liquid, a paste, a semi-solid, or a solid (e.g., a pellet, a granule, or powder), and is frozen, dried, or freeze-dried.

In an embodiment, a colony-forming unit (CFU) ratio of the CCFM1025 strain to the LPL28 strain to the TYCA06 strain is 1:(6 to 20):(2 to 4), preferably 1:(6, 7, 8, 9, 10, 11, 12, 13, 14, 15, 16, 17, 18, 19, or 20):(2, 3, or 4), and more preferably 1:7:2.

Based on the bacterial strain composition can increase enterochromaffin cell number and reduce cortisol level, the bacterial strain composition can be prepared as a medicine, a food, or a health supplement for increasing enterochromaffin cell number and reducing cortisol level. That is, the medicine, the food, or the health supplement can be administered to a subject in need of increasing enterochromaffin cell number and reducing cortisol level. The so-called “subject” indicates any mammal of interest, e.g., a human, a monkey, a cow, a sheep, a horse, a pig, a goat, a dog, a cat, a mouse, or a rat. In an embodiment, the administration is at a total bacterial count of the CCFM1025 strain, the LPL28 strain, and the TYCA06 strain from 10⁶ to 10¹⁰ CFU/kg of body weight of the subject per day, preferably from 10⁷ to 10⁹ CFU/kg of body weight of the subject per day.

Based on the bacterial strain composition can increase enterochromaffin cell number and reduce cortisol level, the medicine, the food, or the health supplement may be further used for treating or preventing insomnia, depression, atypical depression, bipolar disorder, disruptive mood dysregulation disorder, hypertension, anxiety, panic, emotion instability, attention deficit, menopause syndrome, metabolic syndrome, Cushing's syndrome, post-traumatic stress disorder, or gastrointestinal disease, for improving immunity, or for improving sleep disorder.

Additionally, the medicine, the food, or the health supplement may further comprise a physiologically acceptable excipient, diluent, or carrier.

On condition of the food or the health supplement, the bacterial strain composition can be added to the physiologically acceptable excipient, diluent, or carrier to form the food or the health supplement, and the physiologically acceptable excipient, diluent, or carrier may be an edibly acceptable excipient, diluent, or carrier, for example but not limited to a fluid milk (e.g., milk or condensed milk), a fermented milk (e.g., a soured milk), a milk powder, an ice cream, a cheese, a cottage cheese, a soy milk, a fermented soy milk, a vegetable juice, a fruit juice, a sports drink, a jelly, a cookie, an energy bar, a health food, an animal feed, or a dietary supplement.

On condition of the medicine, the bacterial strain composition can be added to the physiologically acceptable excipient, diluent, or carrier to form the medicine, and the physiologically acceptable excipient, diluent, or carrier may be a pharmaceutically acceptable excipient, diluent, or carrier, for example but not limited to a solvent, a buffer agent, an emulsifying agent, a suspending agent, a decomposing agent, a disintegrant, a dispersant, a binder, a stabilizing agent, a chelating agent, a gelling agent, a humectant, a lubricant, an absorption delaying agent, or a liposome. The medicine can be administered to the subject via oral administration, topical administration, or parenteral administration, and the so-called “parenteral administration” comprises but not limited to intraperitoneal injection, intrapleural injection, intramuscular injection, intravenous injection, intraarterial injection, intraarticular injection, intrasynovial injection, intraepidermal injection, subcutaneous injection, intradermal injection, intralesional injection, or sublingual administration.

On condition of oral administration, the medicine can be formulated in the oral dosage form, for example but not limited to a powder, a tablet, a troche, a lozenge, a pellet, a capsule, a dispersible powder, a granule, a solution, a suspension, an emulsion, a syrup, or a slurry. On condition of topical administration, the medicine can be formulated in the external preparation suitable for topical application to skin, for example but not limited to an emulsion, a gel, an ointment, a cream, a patch, a liniment, a powder, an aerosol, a spray, a lotion, a serum, a paste, a foam, a drop, a suspension, a salve, or a bandage. On condition of parenteral administration, the medicine can be in the suitable injectable, for example but not limited to a sterile aqueous solution or a suspension.

The following examples are offered for further illustrating the invention.

### Example 1: Animal Model

Fifty 6- to 8-week-old male C57BL/6 mice are fed in a constant temperature of 22±2° C., a humidity of 50±5%, and a day-night cycle of 12 hrs. During feeding, all mice are with free access to chow and water and divided into five groups after 1 week of adaptation. Each group contain ten mice, and the experiment duration is up to 5 weeks. “the Control group” indicate mice without induction of chronic unpredictable mild stress (CUMS) and without administration of any drug or bacterial strain; “the CUMS group” indicate mice merely with induction of CUMS; “the fluoxetine group” indicate mice with induction of CUMS and with administration of fluoxetine; “the LGG group” indicate mice with induction of CUMS and with administration of Lactobacillus rhamnosus LGG strain; “the bacterial strain composition group” indicate mice with induction of CUMS and with administration of a bacterial strain composition, wherein the bacterial strain composition contains a Bifidobacterium breve CCFM1025 strain, a Lactobacillus plantarum LPL28 strain, and a Lactobacillus acidophilus TYCA06 strain at a CFU ratio of the CCFM1025 strain to the LPL28 strain to the TYCA06 strain as 1:7:2. It is noted that the total CFU of “the LGG group” is the same as the total CFU of “the bacterial strain composition group”.

Chronic unpredictable mild stress is a widely used way for inducing anxiety and depression on rodents and is performed with reference to Psychopharmacology (Berl). 1987; 93(3):358-64. Except for “the control group”, CUMS is performed on the other groups to induce anxiety and depression. CUMS is established with the following stress factors: forced swimming, physical restraint, lack of food and water, change of feeding environment (isolation, wet bedding, lack of bedding, and tilted cage), clamping mouse's tails, and change of irradiation properties (continuous irradiation). These stress factors randomly occur during the experiment so that mice can't expect the occurrence of stimulus.

### Example 2: Bacterial Strain Incubation

All bacterial strains used for efficacy evaluation are deposited at the Guangdong Microbial Culture Center or at the China General Microbiological Culture Collection Center according to the Budapest Treaty. The deposition information of all bacterial strains is listed in Table 1.

TABLE 1
deposition information

| | Deposition | Deposition |
|---|---|---|
| Bacterial strain | number | date |
| Bifidobacterium breve CCFM1025 | GDMCC 60386 | Jun. 11, 2018 |
| Lactobacillus plantarum LPL28 | CGMCC 17954 | Jun. 18, 2019 |
| Lactobacillus acidophilus TYCA06 | CGMCC 15210 | Jan. 15, 2018 |

Lactobacillus rhamnosus LGG strain is purchased from Chr. Hansen A/S, Denmark as control.

Each bacterial strain is seeded onto an MRS agar plate with streaking, and incubated in an incubator (37° C. and 5% CO₂) for 24 hrs to 48 hrs. The bacterial strain is passaged twice for activation. A single colony of the activated CCFM1025 strain is seeded into an MRS broth medium, and single colonies of the activated LGG strain, TYCA strain, and LPL28 strain are seeded into an LBS medium. Then, the strain is incubated in an incubator (37° C. and 5% CO₂). After the late logarithmic phase, the culture is centrifuged in 4° C. with 6000 g for 10 min so that the bacterial body is precipitated. After removing the supernatant, the pellet is suspended with phosphate buffered saline (PBS) and centrifuged with the same condition twice to wash the bacterial body. Finally, the pellet is suspended with 10% skimmed milk solution to obtain a bacterial solution having a concentration of 10′ CFU/mL.

### Example 3: Light/Dark Box Test

The light/dark box test is established based on rodents' ethological property of innate aversion and avoidance to bright illumination. If the number of animal's staying in the light room increases or the number of conversions in the light and dark room increases, the animal shows lower anxiety level.

Mice are placed into the box to face the light room, and the animal behavior trajectory tracking analysis system (EthoVision) is used to detect mouse's activity situation for 5 min. Finally, the ratio of the number of the entries to the light room to the total entries in the box is calculated.

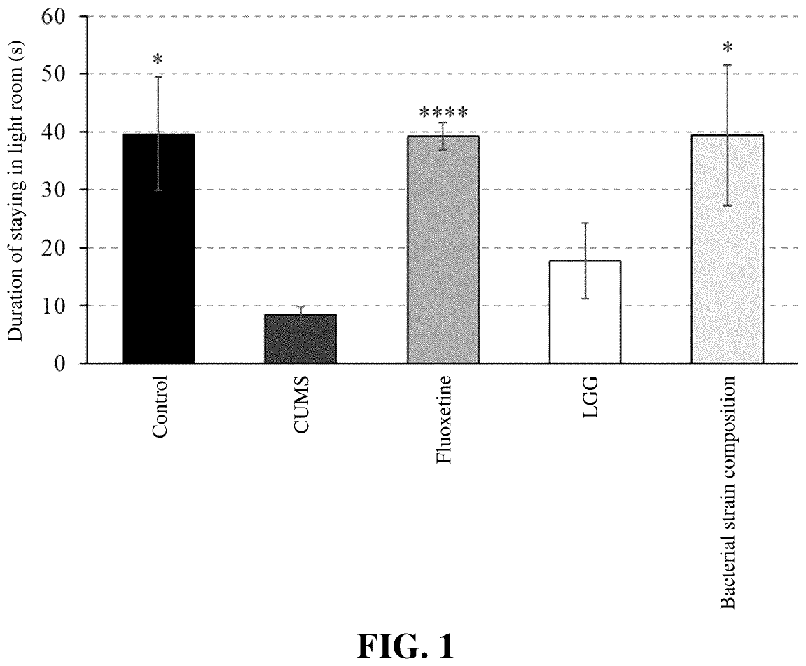

As shown in FIG. 1, the duration of staying in the light room of the CUMS group is lower than that of the control group, which indicates that CUMS successfully induces anxiety and depression on mice. Fluoxetine is a selective serotonin reuptake inhibitor, which can inhibit serotonin reuptake by nerve synapse cells to increase the extracellular level of serotonin for binding to postsynaptic receptors and then can increase the serotonin level in the brain to produce antidepressant effect. Fluoxetine can increase the brain serotonin level to relieve anxiety. As compared with the CUMS group, the duration of staying in the light room of the bacterial strain composition group increases by 4 times, and that of the LGG group merely increases one time. Additionally, the duration of staying in the light room of the bacterial strain composition group is at least two times as high as that of the LGG group. Particularly, the bacterial strain composition can significantly increase the CUMS mouse's duration of staying in the light room to an extent approximately equal to that of the control group, which indicates the CCFM1025 strain, the TYCA strain, and the LPL28 strain can cooperatively and efficiently relieve anxiety behavior and render the behavior normal.

Chromogranin A (CgA) is a protein isolated and identified from chromaffin cells and can be as a biomarker for recognizing chromaffin cells, which is found in Biochem J. 1965 December; 97(3):40C-41C. The antibody specific to CgA is used in immunohistochemistry (IHC) for counting chromaffin cells in mouse's ileum.

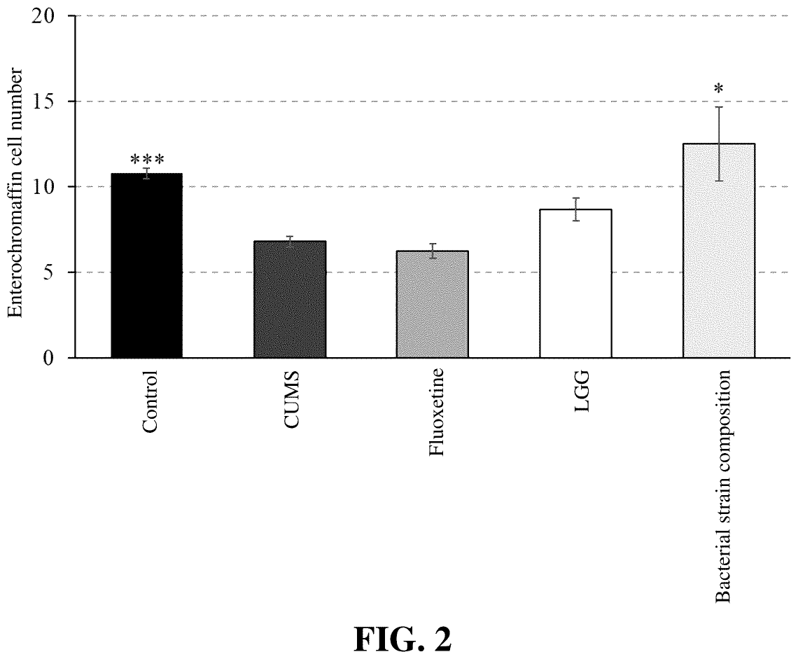

As shown in FIG. 2, the number of enterochromaffin cells in mouse's ileum in the CUMS group decreases. As compared with the CUMS group, the number of enterochromaffin cells in mouse's ileum of the bacterial strain composition group increases by one time, and that of the LGG group slightly increases. Particularly, the bacterial strain composition can significantly increase the number of enterochromaffin cells in mouse's ileum to an extent approximately higher than that of the control group, which indicates the CCFM1025 strain, the TYCA strain, and the LPL28 strain can cooperatively and efficiently increase the number of enterochromaffin cells in mouse's ileum and render the cell number more than normal.

Tryptophan is one of essential amino acids for human and mainly used as the material for brain serotonin. Adequate serotonin in the brain relaxes human's emotion and makes human happy to improve depression and anxiety problems.

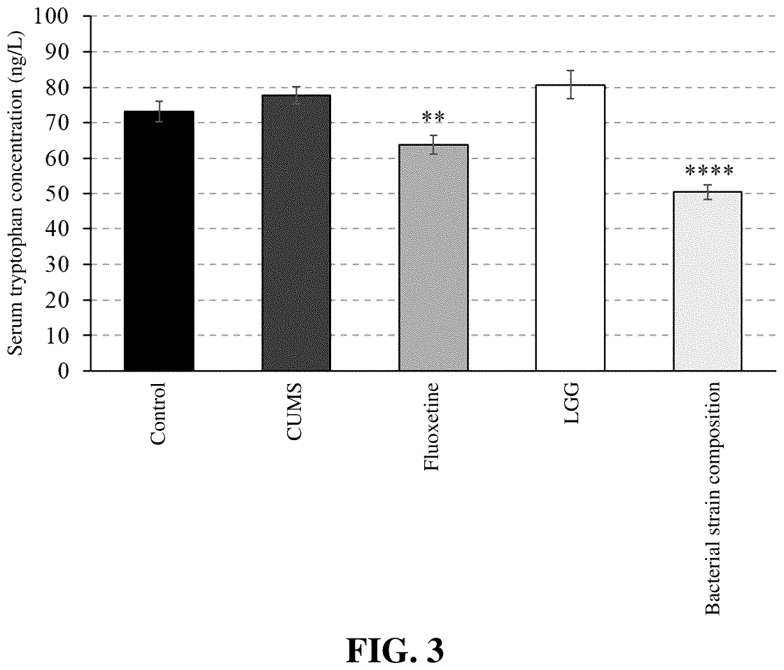

As shown in FIG. 3, as compared with the CUMS group, the bacterial strain composition group can decrease the CUMS mouse's serum tryptophan level by 30%, and the LGG group has a serum tryptophan level close to that of the CUMS group, which indicates the other body tissues can absorb CUMS mouse's serum tryptophan of the bacterial strain composition group so as to lower the tryptophan level in the peripheral circulation. It is learnt from this the CCFM1025 strain, the TYCA strain, and the LPL28 strain can cooperatively and efficiently promote tryptophan absorption in the body tissues of the CUMS mouse.

After dietary tryptophan is absorbed in the intestinal tract, tryptophan hydroxylase 1 (TpH1) in enterochromaffin cells converts tryptophan into 5-hydroxytryptophan, and then L-amino acid decarboxylase (AADC) converts 5-hydroxytryptophan into 5-hydroxytryptamine.

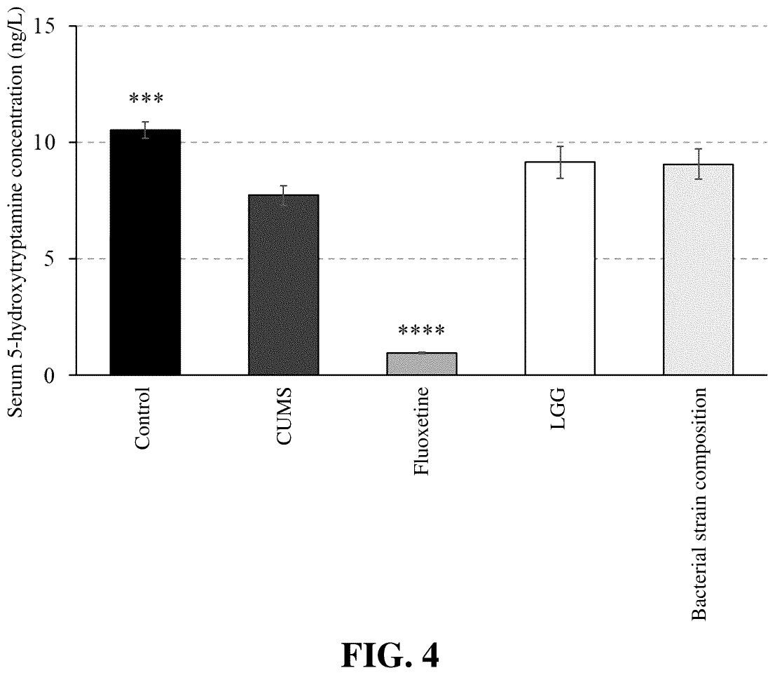

As shown in FIG. 4, the serum 5-hydroxytryptamine level of the control group is higher than that of the CUMS group. Although the LGG group and the bacterial strain composition group can increase the CUMS mouse's 5-hydroxytryptamine level, but the level is still lower than normal, which indicates the CCFM1025 strain, the TYCA strain, and the LPL28 strain relieve CUMS mouse's depression behavior not mainly by increasing the mouse's serum 5-hydroxytryptamine level. Accordingly, there is a need to investigate the CUMS mouse's serum corticosterone level.

Rodent's corticosterone corresponds to human's cortisol. Stress can increase the cortisol level in the body, which facilitate facing stress. Cortisol secretion can decrease the serotonin level to influence emotion.

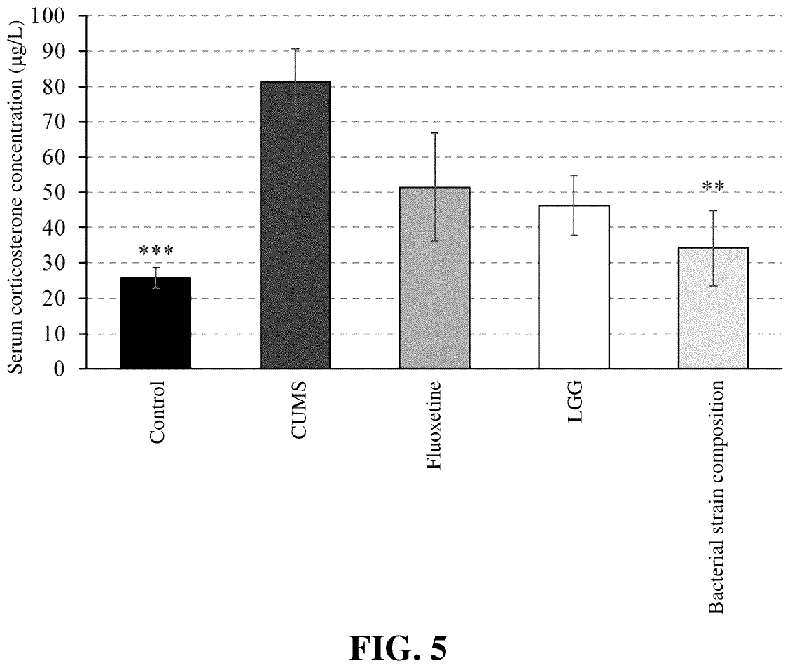

As shown in FIG. 5, the mouse's serum corticosterone level of the control group is lower than that of the CUMS group. As compared with the CUMS group, the bacterial strain composition group can decrease the CUMS mouse's serum corticosterone level by more than 50%, and the decrease of the LGG group is less than 50%, which indicates the CCFM1025 strain, the TYCA strain, and the LPL28 strain can cooperatively and efficiently decrease the mouse's serum corticosterone level and render the level close to normal.

### Example 4: Viability Test on Rat Pancreatic Islet Tumor RIN-14B Cells

1×10⁴ rat pancreatic islet tumor RIN-14B cells are seeded into a 96-well plate. The plate is placed in an incubator (37° C. and 5% CO₂) for 24 hr for cell incubation. After the cells are washed with 150 μL of phosphate buffered saline (PBS) twice, a bacterial suspension is added into each well at a volume of 150 μL, and the total bacterial count therein is 2×10⁸ CFU. Additionally, 150 μL of RPMI-1640 medium (containing 10% fetal bovine serum) is used as a control group. The plate is placed in an incubator (37° C. and 5% CO₂) for 24 hr for cell incubation. After co-incubation, the medium is removed, and the cells are washed with 150 μL of phosphate buffered saline twice. 100 μL of MTT solution (concentration of 5 mg/mL) is added into each well. The plate is placed in an incubator (37° C. and 5% CO₂) for 30 min to 4 hr for reaction. After the MTT solution is removed, 150 μL of dimethyl sulfoxide (DMSO) is added into each well to dissolve the purple crystal, and then shaken in a dark place for 15 min. Absorbance at wavelength 570 nm is measured by a spectrophotometer. The cell viability is calculated according to the following formula:

$$Cell viability = \frac{Absorbance of the experiment group}{Absorbance of the control group} \times 100 \% .$$

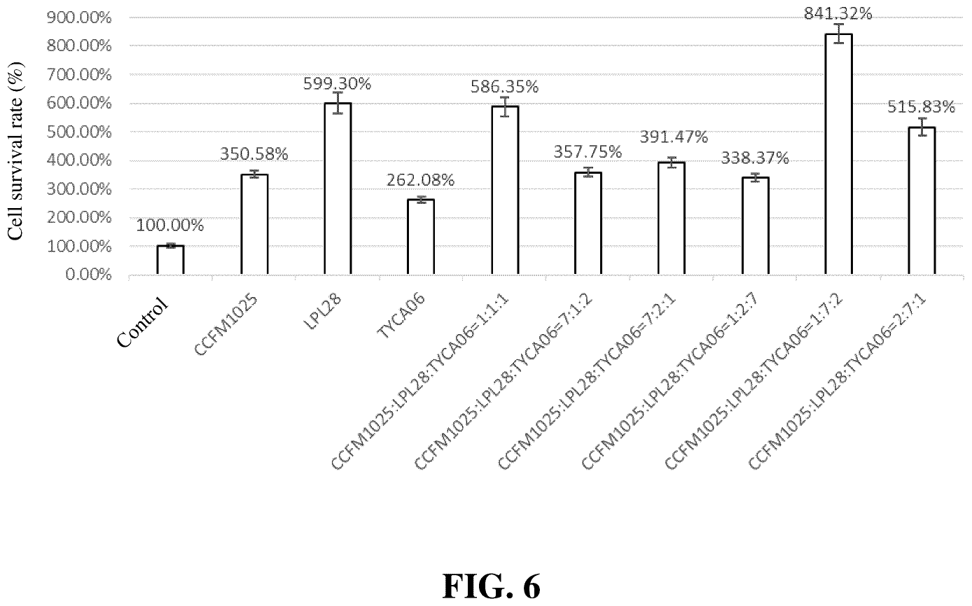

As shown in FIG. 6, “the CCFM1025 group” indicate the added bacterial suspension merely contains the CCFM1025 strain; “the LPL28 group” indicate the added bacterial suspension merely contains the LPL28 strain; “the TYCA06 group” indicate the added bacterial suspension merely contains the TYCA06 strain; “the CCFM1025:LPL28:TYCA06=1:1:1 group” indicate the added bacterial suspension contains the CCFM1025 strain, the LPL28 strain, and the TYCA06 strain at a CFU ratio of the CCFM1025 strain to the LPL28 strain to the TYCA06 strain as 1:1:1; “the CCFM1025:LPL28:TYCA06=7:1:2 group” indicate the added bacterial suspension contains the CCFM1025 strain, the LPL28 strain, and the TYCA06 strain at a CFU ratio of the CCFM1025 strain to the LPL28 strain to the TYCA06 strain as 7:1:2; “the CCFM1025:LPL28:TYCA06=7:2:1 group” indicate the added bacterial suspension contains the CCFM1025 strain, the LPL28 strain, and the TYCA06 strain at a CFU ratio of the CCFM1025 strain to the LPL28 strain to the TYCA06 strain as 7:2:1; “the CCFM1025:LPL28:TYCA06=1:2:7 group” indicate the added bacterial suspension contains the CCFM1025 strain, the LPL28 strain, and the TYCA06 strain at a CFU ratio of the CCFM1025 strain to the LPL28 strain to the TYCA06 strain as 1:2:7; “the CCFM1025:LPL28:TYCA06=1:7:2 group” indicate the added bacterial suspension contains the CCFM1025 strain, the LPL28 strain, and the TYCA06 strain at a CFU ratio of the CCFM1025 strain to the LPL28 strain to the TYCA06 strain as 1:7:2; “the CCFM1025:LPL28:TYCA06=2:7:1 group” indicate the added bacterial suspension contains the CCFM1025 strain, the LPL28 strain, and the TYCA06 strain at a CFU ratio of the CCFM1025 strain to the LPL28 strain to the TYCA06 strain as 2:7:1.

The cell viability of the CCFM1025:LPL28:TYCA06=1:7:2 group (841.32%) is higher than that of the CCFM1025 group (350.58%), the LPL28 group (599.30%), and the TYCA06 group (262.08%). This implies that the survival of rat pancreatic islet tumor RIN-14B cells is synergistically enhanced on the condition of the CFU ratio of the CCFM1025 strain to the LPL28 strain to the TYCA06 strain as 1:7:2. Proc Natl Acad Sci USA. 2009 Mar. 3; 106(9):3408-13 suggests that rat pancreatic islet tumor RIN-14B cells have similar functions to enterochromaffin cells, and thus rat pancreatic islet tumor RIN-14B cells can be used as a model for studying the functions of enterochromaffin cells. That is, the survival of enterochromaffin cells is synergistically enhanced on the condition of the CFU ratio of the CCFM1025 strain to the LPL28 strain to the TYCA06 strain as 1:7:2.

### Example 5: Cortisol Secretion Test on Human Adrenocortical HAC15 Cells

5×10⁵ human colorectal adenocarcinoma Caco-2 cells are seeded into a 6-well plate. The plate is placed in an incubator (37° C. and 5% CO₂) for 48 hr for cell incubation. After the cells are washed with 150 μL of phosphate buffered saline, a bacterial suspension is added into each well at a volume of 3 mL, and the total bacterial count therein is 5×10⁷ CFU. After evenly mixing, the plate is placed in an incubator (37° C. and 5% CO₂) for 24 hr for cell incubation, and then the culture supernatant is collected.

2.5×10⁶ human adrenocortical HAC15 cells are seeded into a 12-well plate. The plate is placed in an incubator (37° C. and 5% CO₂) for 48 hr for cell incubation. After the medium is removed and the cells are washed with 150 μL of phosphate buffered saline, Foskolin is added into each well to induce the cortisol synthesis on the cells and further the collected culture supernatant is added into each well at a volume of 2 mL. After the plate is placed in an incubator (37° C. and 5% CO₂) for 18 hr for cell incubation.

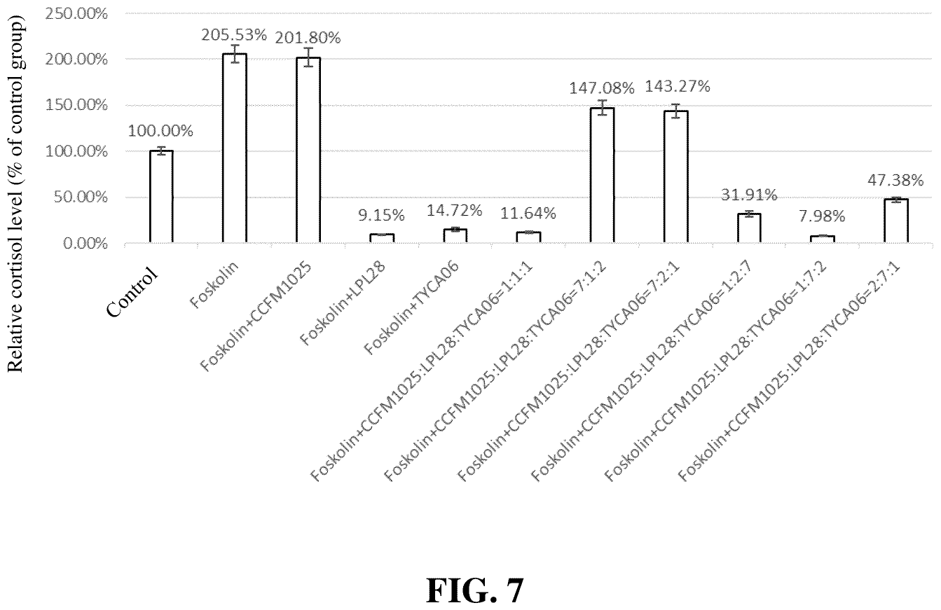

As shown in FIG. 7, “the Foskolin group” indicates no culture supernatant is added when Foskolin is added; “the Foskolin+CCFM1025 group” indicate the bacterial suspension merely contains the CCFM1025 strain, and the corresponding culture supernatant is added when Foskolin is added; “the Foskolin+LPL28 group” indicate the bacterial suspension merely contains the LPL28 strain, and the corresponding culture supernatant is added when Foskolin is added; “the Foskolin+TYCA06 group” indicate the bacterial suspension merely contains the TYCA06 strain, and the corresponding culture supernatant is added when Foskolin is added; “the Foskolin+CCFM1025:LPL28:TYCA06=1:1:1 group” indicate the bacterial suspension contains the CCFM1025 strain, the LPL28 strain, and the TYCA06 strain at a CFU ratio of the CCFM1025 strain to the LPL28 strain to the TYCA06 strain as 1:1:1, and the corresponding culture supernatant is added when Foskolin is added; “the Foskolin+CCFM1025:LPL28:TYCA06=7:1:2 group” indicate the bacterial suspension contains the CCFM1025 strain, the LPL28 strain, and the TYCA06 strain at a CFU ratio of the CCFM1025 strain to the LPL28 strain to the TYCA06 strain as 7:1:2, and the corresponding culture supernatant is added when Foskolin is added; “the Foskolin+CCFM1025:LPL28:TYCA06=7:2:1 group” indicate the bacterial suspension contains the CCFM1025 strain, the LPL28 strain, and the TYCA06 strain at a CFU ratio of the CCFM1025 strain to the LPL28 strain to the TYCA06 strain as 7:2:1, and the corresponding culture supernatant is added when Foskolin is added; “the Foskolin+CCFM1025:LPL28:TYCA06=1:2:7 group” indicate the bacterial suspension contains the CCFM1025 strain, the LPL28 strain, and the TYCA06 strain at a CFU ratio of the CCFM1025 strain to the LPL28 strain to the TYCA06 strain as 1:2:7, and the corresponding culture supernatant is added when Foskolin is added; “the Foskolin+CCFM1025:LPL28:TYCA06=1:7:2 group” indicate the bacterial suspension contains the CCFM1025 strain, the LPL28 strain, and the TYCA06 strain at a CFU ratio of the CCFM1025 strain to the LPL28 strain to the TYCA06 strain as 1:7:2, and the corresponding culture supernatant is added when Foskolin is added; “the Foskolin+CCFM1025:LPL28:TYCA06=2:7:1 group” indicate the bacterial suspension contains the CCFM1025 strain, the LPL28 strain, and the TYCA06 strain at a CFU ratio of the CCFM1025 strain to the LPL28 strain to the TYCA06 strain as 2:7:1, and the corresponding culture supernatant is added when Foskolin is added.

The relative cortisol level of the Foskolin group (205.53%) is higher than that of the control group (100%), which proves that Foskolin can induce the cells to produce cortisol. The relative cortisol level of the Foskolin+CCFM1025:LPL28:TYCA06=1:7:2 group (7.98%) is lower than that of the Foskolin+CCFM1025 group (201.80%), the Foskolin+LPL28 group (9.15%), and the Foskolin+TYCA06 group (14.72%), which indicates the cell cortisol level is synergistically decreased on the condition of the CFU ratio of the CCFM1025 strain to the LPL28 strain to the TYCA06 strain as 1:7:2.

### Example 6: Statistical Analysis

All experimental data are expressed as “mean±standard deviation”. Differences between experimental data are assessed using the Student's t-test. A p-value of lower than 0.05 is considered statistically significant.

As described above, a composition made by mixing a Bifidobacterium breve CCFM1025 strain, a Lactobacillus plantarum μLPL28 strain, and a Lactobacillus acidophilus TYCA06 strain has a positive effect on the properties of increasing enterochromaffin cell number and reducing cortisol level. Therefore, the composition is expected to be used for improving insomnia, depression, anxiety, emotion instability, attention deficit, menopause syndrome, metabolic syndrome, Cushing's syndrome, post-traumatic stress disorder, or gastrointestinal disease.

While the invention has been described in connection with what is considered the most practical and preferred embodiments, it is understood that this invention is not limited to the disclosed embodiments but is intended to cover various arrangements included within the spirit and scope of the broadest interpretation so as to encompass all such modifications and equivalent arrangements.

What is claimed is:

1. A bacterial strain composition for increasing enterochromaffin cell number and reducing cortisol level, comprising:
a Bifidobacterium breve CCFM1025 strain, a Lactobacillus plantarum LPL28 strain, and a Lactobacillus acidophilus TYCA06 strain;
wherein the Bifidobacterium breve CCFM1025 strain is deposited at the Guangdong Microbial Culture Center with a deposition number of GDMCC 60386; the Lactobacillus plantarum μLPL28 strain is deposited at the China General Microbiological Culture Collection Center with a deposition number of CGMCC 17954; the Lactobacillus acidophilus TYCA06 strain is deposited at the China General Microbiological Culture Collection Center with a deposition number of CGMCC 15210.

2. The bacterial strain composition as claimed in claim 1, wherein the Bifidobacterium breve CCFM1025 strain is a viable bacterial strain or a deactivated bacterial strain; the Lactobacillus plantarum μLPL28 strain is a viable bacterial strain or a deactivated bacterial strain; the Lactobacillus acidophilus TYCA06 strain is a viable bacterial strain or a deactivated bacterial strain.

3. The bacterial strain composition as claimed in claim 1, wherein a colony-forming unit (CFU) ratio of the Bifidobacterium breve CCFM1025 strain to the Lactobacillus plantarum μLPL28 strain to the Lactobacillus acidophilus TYCA06 strain is 1:(6 to 20):(2 to 4).

4. The bacterial strain composition as claimed in claim 1, wherein a colony-forming unit (CFU) ratio of the Bifidobacterium breve CCFM1025 strain to the Lactobacillus plantarum μLPL28 strain to the Lactobacillus acidophilus TYCA06 strain is 1:7:2.

5. A method for increasing enterochromaffin cell number and reducing cortisol level, comprising:
administering to a subject in need thereof a composition comprising: a Bifidobacterium breve CCFM1025 strain, a Lactobacillus plantarum μLPL28 strain, and a Lactobacillus acidophilus TYCA06 strain;
wherein the Bifidobacterium breve CCFM1025 strain is deposited at the Guangdong Microbial Culture Center with a deposition number of GDMCC 60386; the Lactobacillus plantarum μLPL28 strain is deposited at the China General Microbiological Culture Collection Center with a deposition number of CGMCC 17954; the Lactobacillus acidophilus TYCA06 strain is deposited at the China General Microbiological Culture Collection Center with a deposition number of CGMCC 15210.

6. The method as claimed in claim 5, wherein the composition is a medicine, a food, or a health supplement.

7. The method as claimed in claim 5, wherein a colony-forming unit (CFU) ratio of the Bifidobacterium breve CCFM1025 strain to the Lactobacillus plantarum LPL28 strain to the Lactobacillus acidophilus TYCA06 strain is 1:(6 to 20):(2 to 4).

8. The method as claimed in claim 5, wherein a colony-forming unit (CFU) ratio of the Bifidobacterium breve CCFM1025 strain to the Lactobacillus plantarum LPL28 strain to the Lactobacillus acidophilus TYCA06 strain is 1:7:2.

9. The method as claimed in claim 5, wherein the Bifidobacterium breve CCFM1025 strain is a viable bacterial strain or a deactivated bacterial strain; the Lactobacillus plantarum μLPL28 strain is a viable bacterial strain or a deactivated bacterial strain; the Lactobacillus acidophilus TYCA06 strain is a viable bacterial strain or a deactivated bacterial strain.

10. The method as claimed in claim 5, being further for treating or preventing insomnia, depression, atypical depression, bipolar disorder, disruptive mood dysregulation disorder, hypertension, anxiety, panic, emotion instability, attention deficit, menopause syndrome, metabolic syndrome, Cushing's syndrome, post-traumatic stress disorder, or gastrointestinal disease, for improving immunity, or for improving sleep disorder.

11. The method as claimed in claim 5, wherein the administration is at a total bacterial count of the Bifidobacterium breve CCFM1025 strain, the Lactobacillus plantarum μLPL28 strain, and the Lactobacillus acidophilus TYCA06 strain from 10⁶ to 10¹¹ CFU/kg of body weight of the subject per day.
